(19) United States
(12) Patent Application Publication
(10) Pub. No.: US 2023/0340165 A1
(43) Pub. Date: Oct. 26, 2023

(54) CROSS-LYNKED HYALURONIC ACID SYNTHESIS PROCESS

(71) Applicant: ISTITUTO GANASSINI S.P.A. DI RICERCHE BIOCHIMICHE, Milano (IT)

(72) Inventors: Stefano Manfredini, Ferrara (IT); Silvia Vertuani, Ferrara (IT); Sabrina Sciabica, Ferrara (IT); Anna Baldisserotto, Ferrara (IT); Luisa Canilli, Milano (IT); Massimo Terno, Milano (IT)

(21) Appl. No.: 17/785,711

(22) PCT Filed: Dec. 16, 2020
(86) PCT No.: PCT/IB2020/062024

§ 371 Date: Jun. 15, 2022

(30) Foreign Application Priority Data
Dec. 16, 2019 (IT) 102019000024117

(51) Int. Cl. C08B 37/08; B01J 31/02

(52) U.S. Cl. CPC C08B 37/0072; B01J 31/0244

(57) ABSTRACT

The present invention refers to a synthesis process of cross-linked hyaluronic acid, cross-linked hyaluronic acid, a cross-linking agent and the topical or systemic use of cross-linked hyaluronic acid.

### TECHNICAL FIELD OF THE INVENTION

The present invention refers to a synthesis process of cross-linked hyaluronic acid.

### STATE OF THE ART

Hyaluronic acid or HA is a viscoelastic biomaterial composed of repeating units of D-glucuronic acid and N-acetylglucosamine joined through β 1-3 glycosidic bonds to form a repeating unit of linear chain disaccharide, linked by β 1-4 glycosidics bonds. It is a heteropolymer constitutively present in the soft tissues of many vertebrates where it plays fundamental roles linked to its chemical-physical and biological properties. Thanks to its natural ability to coordinate many water molecules, it reaches a high degree of hydration and forms highly viscous matrices, depending on the molecular weight and concentration, which impart consistency, elasticity and lubricating capacity in various tissues such as connective tissue, liquid synovial and vitreous humor of the eye.

The properties of this polymer therefore make it an ideal candidate for the synthesis of new biopolymer derivatives, of application in various clinical fields both as a filler for wrinkles, folds and scars in cosmetic applications and as a regenerating of the skin tissue and healing, being a polymer with low risk and well tolerated.

Many of the potential applications are limited by the fact that hyaluronic acid is highly soluble in an aqueous environment in its natural state and has a rapid turnover through enzymatic and chemical metabolization. After interaction with the CD44 receptor present on the surface of the cell membrane, it is internalized and rapidly degraded by specific enzymes, hyaluronidases (HAse), evoking a clear reduction in the half-life of the molecule due to the degradation to small fragments of polymer.

Another chemical degradation mechanism, but of a non-specific type, occurs due to the effect of free radicals, as well as reactive oxygen species (ROS), produced massively during inflammatory responses and in the case of tissue injury. It is possible to slow down this degradation process by generally resorting to modifications of linear hyaluronic acid to protect the glycosidic bonds.

The idea of crosslinking native hyaluronic acid starts from this need, through the use of small molecules defined as crosslinkers such as epoxides, dihydrazides and divinylsulfones. The hyaluronic acid polymer cross-linked with these cross-linking agents is less susceptible to the enzymatic action of hyaluronidases, since the modification reduces the solubility in water with a consequent increase in permanence in situ.

However, the common use of the aforementioned agents appears to be poorly biocompatible with the body's tissues, causing the onset of adverse reactions related to cellular toxicity.

### OBJECTS OF THE INVENTION

Therefore, the object of the present invention is to provide a new process for the synthesis of cross-linked hyaluronic acid.

Another object of the present invention is to provide a process as indicated above which is capable of producing hyaluronic acid which guarantees a prolonged half-life of the latter when injected into the site of use.

Another purpose of the present invention is to provide a process as above which allows to obtain cross-linked hyaluronic acid which is biocompatible in a highly manner with the body tissues of the implantation or injection.

A further object is to obtain a cross-linked hyaluronic acid having mechanical and viscoelastic properties useful for use in the cosmetic, nutraceutical and medical device sectors.

Another object of the present invention is to provide a new use of hyaluronic acid.

### BRIEF DESCRIPTION OF THE DRAWINGS

FIG. 1 schematically shows the transformation undergone by hyaluronic acid during the execution of the process according to the present invention,

FIGS. 2, 3 and 4 show, respectively, the ¹H-NMR spectra of linear sodium hyaluronate, of sodium hyaluronate cross-linked with arginine and of sodium hyaluronate cross-linked with ornithine,

FIGS. 5 to 7 show, respectively, IR spectra of linear HA, HA cross-linked with arginine and HA cross-linked with ornithine,

FIGS. 8 to 10 show, respectively, the thermal behaviour of linear HA, of HA cross-linked (HA-CL) with arginine and of HA cross-linked with ornithine (HA-CL-ORN),

FIG. 11 shows a graph indicating the swelling degree of linear hyaluronic acid (HA) and cross-linked hyaluronic acid (HA-CL) at different pH values,

FIGS. 12 to 14 show the rheological analysis conducted on a sample of 2% cross-linked HA under conditions of continuous flow and oscillatory flow, amplitude sweep and frequency sweep, respectively,

FIGS. 15 to 20 show SEM images of linear HA (A and C), HA cross-linked with arginine (B and D) and HA cross-linked with ornithine (E and F).

### EMBODIMENTS OF THE INVENTION

According to the present invention, a cross-linked hyaluronic acid synthesis process is therefore provided which comprises the following steps:
provide a predetermined quantity of hyaluronic acid and of at least one cross-linking agent of an amino acidic nature, preferably in a ratio of 1 to 1.5, the hyaluronic acid being in the form of a linear polymer or even already partially cross-linked, for example with a degree of cross-linking between 10 and 80%, wanting between 20% and 40% or between 60% and 80%, and
cross-link the hyaluronic acid by the at least one cross-linking agent by means of amide bonds operated on the carboxylic portions of linear chains of the linear polymer or of the already partially cross-linked polymer.

The reference to cross-linked hyaluronic acid and cross-linking steps in the present description will be understood with reference to a cross-linking obtained following the process in accordance with the present invention and not before it.

Advantageously, starting from a linear hyaluronic acid with 0% crosslinking degree, thanks to a process according to the present invention it is possible to reach a crosslinking degree greater than 80% or 90%, for example equal to 100%.

If, on the other hand, one starts from partially cross-linked hyaluronic acid (for example with a cross-linking degree between 10 and 80% obtained with any cross-linking agent, for example arginine, ornithine or their derivatives), thanks to a process according to the present invention it is possible to reach a degree of crosslinking greater than 90%, for example equal to 100%.

Moreover, if one uses or starts from partially cross-linked hyaluronic acid to carry out a process according to the present invention, the new amide bonds that will be developed during the latter will involve the non-derivatized carboxylic groups in the first cross-linking step, since the first crosslinking or initial crosslinking was partial.

The obtained products have high biocompatibility linked to the use of cross-linking agents of an amino acid nature that also have healthy properties, that is, which perform useful functions for maintaining the body in a good state of health.

The process is therefore focused on the synthesis of new hyaluronic acid derivatives and more specifically new cross-linking products that exploit one or more biologically active molecules as cross-linking agents, which is/are compatible with the tissues of the human organism, i.e. which have already been shown to have no toxicity or side reactions that provoke a response of the immune system, and with a multidentate structure, for example tridentate, where multidentate is understood to have 2, 3, 4 or more functionalizable portions.

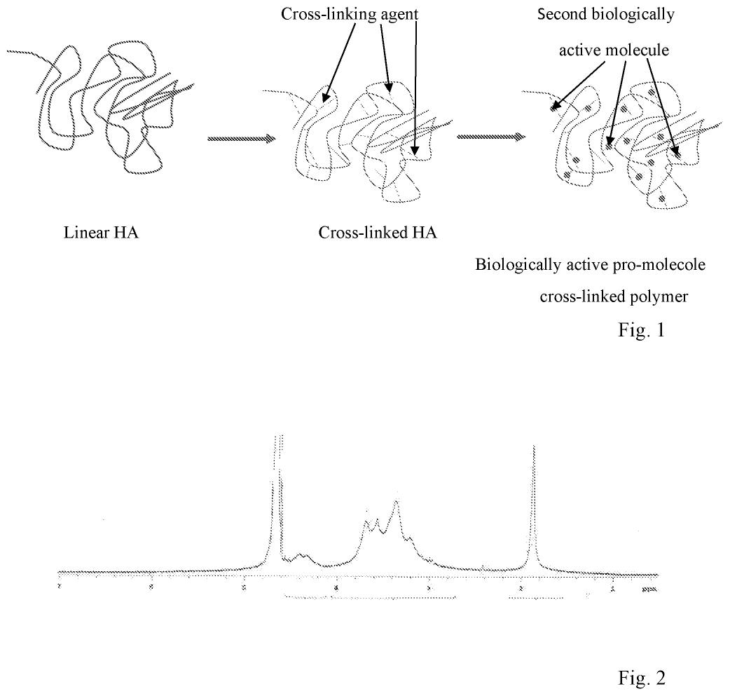

In this regard, the crosslinking or the constitution of a polymeric lattice employs or is carried out by means of the homobifunctional portion of the molecule given by 2 free or salified amino groups, one of which is terminal of the side chain (o) and the other in alpha (a). If a third function is envisaged, it is the carboxyl group, which constitutes a connector or linker of further active molecules that will be inserted into the construct (see in particular FIG. 1).

If desired, the first biologically active molecule comprises at least one amino acid or a substrate for synthesis thereof, for example at least one substrate for proline synthesis.

Advantageously, the first biologically active molecule is selected from the group consisting of arginine, ornithine or their mixtures, for example arginine and/or ornithine methyl ester.

The following is the general formula of a tridentate agent, and in particular of ornithine and arginine among the tridentates, which has now been realized and which can be used in a process according to the present invention.

Arginine and ornithine are to be considered in all respects tridentate agents. In fact, they possess two amino groups capable of forming the cross-link between two chains of HA and a carboxylic function (third “tooth”) remains free, capable of forming covalent or saline bonds with other active molecules, retaining them within the structure.

Another example of tridentate agent, in a non-limiting version of the invention, is 1,3-diamino-2-propanol, or 1,3-diamino-2-propionic acid.

This agent is the subject of the present invention also as such and therefore independently of the process according to the present invention.

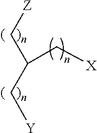

X═—OH, —NH2, —NRH, —SH, —COOH
Y═—OH, —NH2, —NRH, —SH, —COOH
Z═—OH, —NH2, —NRH, —SH, —COOH
R=alkyl, aryl, —NH2, —NHR, —CO-Q
Q=R, —OH, —SH

wherein R is a generic radical and n indicates an n^{th} integer number of possible groups and/or atoms (for example —CH2-, —CHR, —CRR—, —O—, —NRR—, —S—, etc.).

The choice between molecules of this type (substrate of proline synthesis) is given not only by the structural characteristics sought but also by the intrinsic properties they possess, being able to combine them synergistically with the activity of the polymer. Arginine and ornithine show physiological immunomodulating, moisturizing and elasticising activity as well as a structural function, being a substrate for the synthesis of proline, an amino acid abundant in collagen.

Preferably, the first biologically active molecule acts both as a crosslinker, to form cross-links between different linear hyaluronic acid chains or possibly between two different points of the same chain, and as a linker (see FIG. 1), for the constitution of a prodrug as a result of a covalent or saline bond to a further second biologically active molecule. This further second biologically active molecule is capable of forming a covalent or saline bond with the linker, and is for example a drug, for example aspirin, penicillin, steroid, etc., or a healthy ingredient, such as a cosmetic ingredient, for example vitamin E, vitamin C, bisabolol, polyphenols such as gallic acid and resveratrol, etc., or a food ingredient, such as for example sugars, amino acids, lipids, and if desired it is equipped with specific physiological functions, such as soothing, anti-wrinkle, anti-aging, moisturizing, antibacterial, emollient, conditioning, elasticizing, nourishing, colouring, depigmenting, antioxidant, abrasive, UV filtering, deodorant, anaesthetic, antidepressant, anticonvulsant.

It is therefore possible to obtain a multiple action product since, in addition to offering the structuring and moisturizing action typical of hyaluronic acid-based derivatives, it has a better structural solidity given by the increase in the internal organization of the polymer. This makes it a new product that is more stable and resistant to the action of digestive enzymes.

The improved resistance to enzymatic degradation increases the half-life in vivo and translates it into a better bioavailability of both hyaluronic acid and biologically active compounds, cross-linked to it.

Moreover, it is possible to include or disperse at least a second biologically active molecule comprising a drug, that is to say a pharmacologically active molecule or a healthy molecule (intended as such a molecule having positive effects on maintaining good state of health, for example a vitamin) or a cosmetic ingredient or a food ingredient in the polymeric matrix, such as the same molecules indicated above which could be linked to ornithine and arginine, although in this case not linked to the same, but only included in the matrix. In this case, at least a second biologically active molecule, comprising a drug such as a pharmacologically active molecule or a healthy molecule (understood as such a molecule having positive effects on maintaining good state of health, for example a vitamin) or a cosmetic ingredient or a food ingredient, would be housed in the cavities of defined size created with the crosslinking process in order to obtain a prolonged release due to the slowed degradation brought about by the crosslinking itself.

The at least one second biologically active molecule, comprising a drug, such as for example a pharmacologically active molecule or a healthy molecule (understood as such a molecule having positive effects on maintaining good state of health, for example a vitamin) or a cosmetic ingredient or a food ingredient, may be included before or after the crosslinking process and will be released from it following enzymatic or chemical degradation.

The at least one second biologically active molecule, comprising a drug such as a pharmacologically active molecule or a healthy molecule (understood as such a molecule having positive effects on maintaining good state of health, for example a vitamin) or a cosmetic ingredient or ingredient food, can therefore be physically included in the cages (cavities) not only chemically bound.

In this regard, the crosslinking agent is derivatized through free functionality (—COOH for example) with a bioactive molecule as a result of a covalent bond, be it ester or amide or similar, resulting in the formation of a polymeric prodrug.

For this purpose, the second biologically active molecule, preferably, contains in its structure a functional group capable of reacting with the carboxylic group (—COOH for example) of the first linker molecule (e.g. amino acid), such as for example NH2, OH, COOH, NHR, SH, CO—NH2, CO—NHR, NH—NH2, wherein R has the above-mentioned meaning.

The second biologically active molecule must be capable of carrying out a specific biological activity when in free form. Only when the second biologically active molecule is covalently linked to the amino acid—or to the first biologically active molecule—is it defined as a prodrug.

The constitution of the prodrug can take place before or after the crosslinking process.

The second biologically active molecule will remain protected inside the polymeric matrix and only after the degradation of the latter, if desired by the action of the amidases that split the cross-linking bonds or hyaluronidases that split the glycosidic bonds between the linear dimers of the polymer, it will it be exposed to specific hydrolytic activities able to make it free to reach a predefined biological target.

As regards the crosslinking of hyaluronic acid, since the aim is to synthesize a product with a high degree of crosslinking, various reactions have been tried.

In this regard, according to a first reaction, before crosslinking, an initial step of activation of the hyaluronic acid is envisaged by amidation or derivatization with EDC, i.e. 1-ethyl-3-[3-(dimethylamino)-propyl]-carbodiimide.

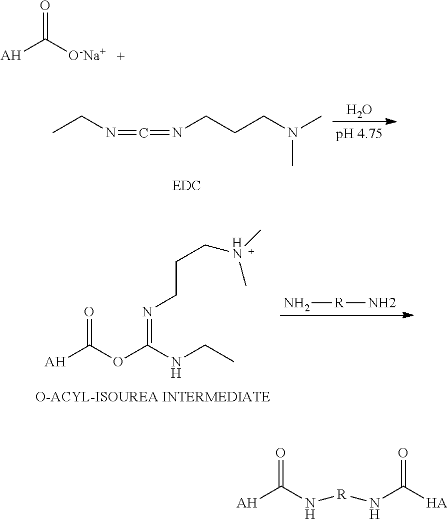

In detail, as shown in the diagram above, the mechanism of this reaction begins with the activation by EDC of the carboxyl group of hyaluronic acid so as to form a highly reactive intermediate. This intermediate would tend to rapidly reorganize into a stable by-product to the point of avoiding the nucleophilic attack of the amine. To this end, it is possible to resort to the use of N-hydroxy-succinimide (NHS) or 1-hydroxybenzotriazole (HOBt) which effectively prevent this reorganization. Preferably, the ratio of the reactants in this reaction is 1:4:4 between hyaluronic acid, EDC/NHS and crosslinking agent.

This activation reaction is also characterized by its strong dependence on pH, since the optimal values required in the various steps are very different, so defining a pH compromise value is very important.

In this regard, the activation step is preferably carried out in water until an ester intermediate is obtained which will undergo the nucleophilic attack of the linker molecule which takes place under optimal conditions at a pH between about 4 and 5, for example at 4.75.

In accordance with a second reaction, before crosslinking, an initial step of activation of hyaluronic acid is envisaged by amidation or derivatization with CDMT, i.e. 2-chloro-dimethoxy-1,3,5-triazine.

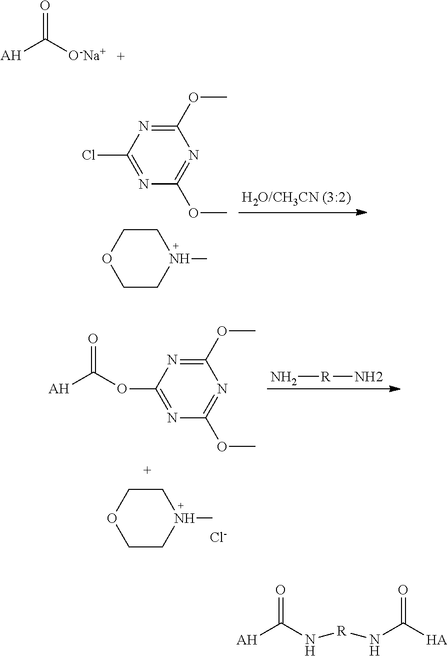

This reaction is carried out in a mixture of water and acetonitrile solvents, for example in a 3:2 ratio for optimal solubilization of the reactants. This amidation reaction in mixture with water can include the addition of N-methylmorpholine (NMN) to neutralize the chloride ions released, for example in a molar ratio 1:3:3:1.5 between hyaluronic acid, triazine and N-methyl-morpholine and cross-linking agent. The hyaluronic intermediate activated with CMDT undergoes the nucleophilic attack of the amino group of the first molecule with consequent formation of the amide bond.

The initial step of activation of hyaluronic acid can alternatively be carried out by derivatization with DMTMM, namely 4-(6-dimethoxy-1,3,5-triazin-2-yl)-4-methyl morpholin chloride.

Several crosslinking tests were carried out using arginine or ornithine or their derivatives as crosslinking agent on the carboxylic group not involved in the crosslinking process such as esters and amides with the activation reactions indicated above and the following table shows the preparation methods and stoichiometric ratios between HA (hyaluronic acid), activating agent and cross-linking agent as well as the respective degrees of cross-linking of the obtained hyaluronic acid.

TABLE 1
Summary of Ha cross-linked preparation tests

| | Cross- | [HA]:[Activating | | Cross- |
|---|---|---|---|---|
| Activating | linking | agent]:[Cross- | [HA] | linking |
| agent | agent | linking agent] | mg/ml | degree |
| EDC/NHS | ARGININE | 1:4:4 | 3 | 25 |
| EDC/NHS | ARGININE | 1:2:4 | 3 | 16 |
| EDC/NHS | ARGININE | 1:1:2 | 3 | NO |
| | | | | product |
| EDC/NHS | ORNITHINE | 1:4:4 | 3 | 32 |
| EDC/NHS | ORNITHINE | 1:2:4 | 3 | 25 |
| EDC/NHS | ORNITHINE | 1:1:2 | 3 | NO |
| | | | | product |
| CDMT/NMM | ARGININE | 1:3:4 | 3 | 40 |
| CDMT/NMM | ARGININE | 1:2:4 | 3 | 55 |
| CDMT/NMM | ARGININE | 1:3:1.5 | 3 | 63 |
| CDMT/NMM | ORNITHINE | 1:3:4 | 3 | 40 |
| CDMT/NMM | ORNITHINE | 1:2:4 | 3 | 65 |
| CDMT/NMM | ORNITHINE | 1:3:1.5 | 3 | 72 |

As it will be possible to ascertain, the best degree of crosslinking has been obtained following the synthetic process that uses CDMT as activating agent in a ratio [HA]:[Activating agent]:[Crosslinking agent] of 1:3:1.5 from which it was possible to obtain easily and reproducibly the desired product of cross-linked HA (HA-CL) with arginine with a percentage degree of cross-linking of 63% and of cross-linked HA (HA-CL) with ornithine of 72%.

Regarding the crosslinking test results using DMTMM as activating agent, since DMTMM is the product of a spontaneous reaction between CDMT and NMM available on the market, the resulting crosslinking products using DMTMM are similar to those obtained with the use of CDMT/NMM added in reaction separately.

The crosslinking products were then evaluated with the analytical techniques deemed suitable and discussed below.

The following are the 3 reaction schemes that illustrate the synthetic paths followed for the synthesis of the crosslinking product (Scheme 1), a) the derivatization of the crosslinking agent b) its use for crosslinking (Scheme 2) as well as for the derivatization of the crosslinking agent post-crosslinking (Scheme 3).

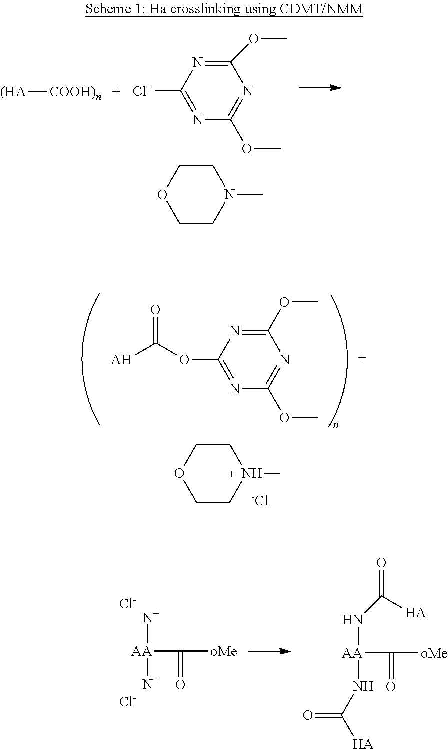

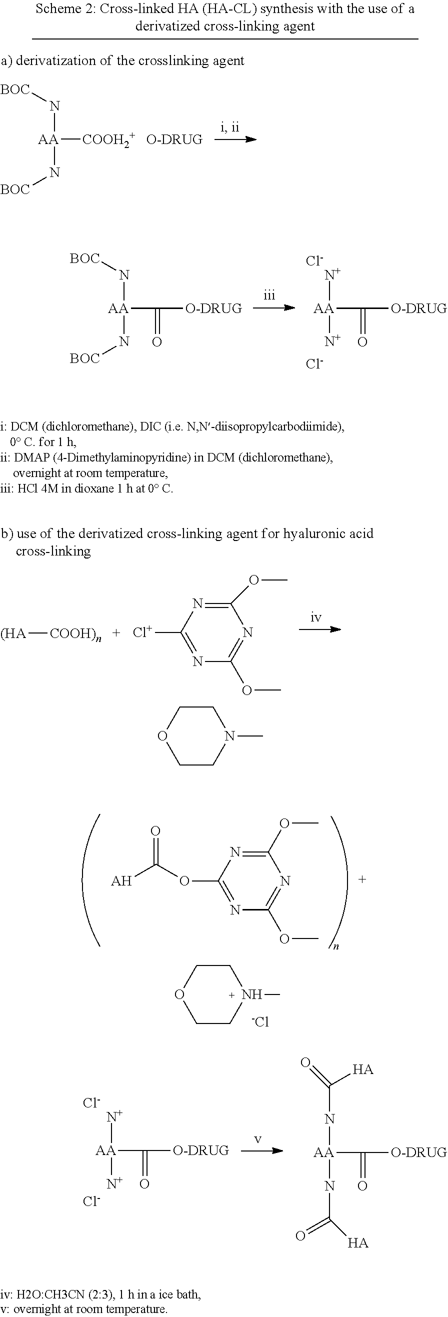

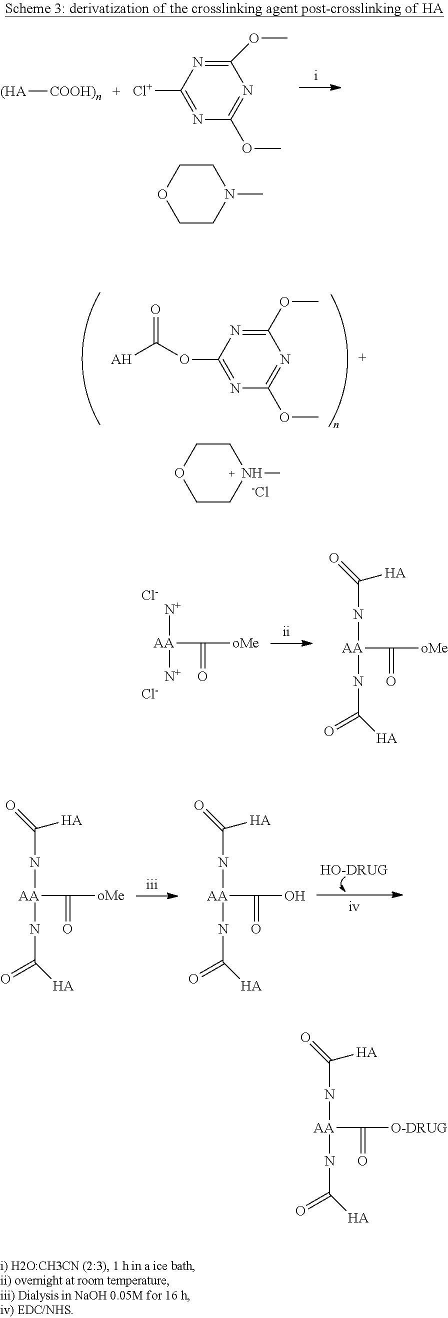

### Example of Cross-Linked Hyaluronic Acid Synthesis Process

A specific embodiment of a cross-linked hyaluronic acid (HA-CL) synthesis process with Arginine-OMe (for the production of a product A) or Ornithine-OMe (for the production of a product B) will now be described in detail using CDMT/NMMN.

As already indicated above, HA crosslinking requires the use of protected amino acids on the carboxylic portion to prevent the formation of amino acid dimers or unwanted reaction products. Therefore, an arginine and an ornithine methyl ester were used for this reaction step, which are easily hydrolysed in mild acidic or basic conditions.

Sodium hyaluronate (for example 264 mg, 0.66 mmol) was initially solubilized in water (for example 52 ml) and left under stirring until a solution clear and free of agglomerated was obtained. Subsequently acetonitrile was added, if desired drop by drop (for example 35 ml) to obtain a solution, if desired, at 0.3% in a mixture of H2O:CH3CN, for example in the ratio 3:2. This solution was then cooled, for example by placing it in an ice bath for 30 minutes and, once cold, the activating agent was introduced, if desired CDMT in solid form (for example 47.6 mg, 1.98 mmol), leaving what is obtained under stirring, for example magnetic stirring, for 1 hour.

After the indicated time, the crosslinking agent A) Arg-OMe (for example 258.54 mg, 0.99 mmol) or B) Orn-OMe (for example 219.12 mg, 0.99 mmol) and then NMM (for example 217 μl, 1.98 mmol) to neutralize chloride ions. The reaction mixture was left under stirring for a time greater than 6 hours, if desired overnight until complete crosslinking and then purified, in particular from amines and excess reagents.

This purification can for example be obtained through a dialysis process consisting in the introduction of the reaction mixture inside a semi-synthetic cellulose-based membrane (3.5 KDa cut-off) sequentially immersed in H2O for a period of 24 h, NaCl 0.1M 40 h and finally 72 h in H2O. The passage in these solvents allows a swelling of the resulting cross-linked polymer and therefore the removal of the molecules not included in the product.

The purified solutions could then be lyophilized and stored at a temperature of about 2-15° C., for example 8° C., isolated from humid environments.

The scheme of this process is in fact the scheme 1 indicated above.

### Example of Derivatization of the Crosslinking Agent

An example of derivatization of the cross-linking agent for the synthesis of cross-linked hyaluronic acid in order to form a prodrug of arginine (for the production of a product C) or ornithine (for the production of a product D) is described in detail below.

The process of derivatization of the crosslinking agent and therefore the formation of the prodrug, as previously mentioned, is a step that can be carried out before or after the HA crosslinking.

A process is reported here in which the derivatization of the amino acid is carried out as the primary step followed by the second cross-linking step through the above procedure. The scheme of this process is in fact the scheme 2 indicated above.

To derivatize the carboxylic group and avoid secondary reactions, the same amino acids are used but protected, in this case, on the amino functionalities with the tert-butyloxycarbonyl group Boc, easily hydrolysable in an acid environment. In the case reported, the crosslinking agent was esterified with Lipocrhoman®, which has the following formula:

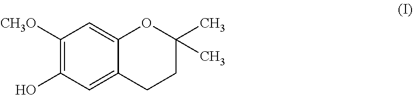

Lipocrhoman® is a semi-synthetic compound, analogue of vitamin E, with a strong antioxidant capacity as it is capable of capturing reactive nitrogen species (RNS) and reactive oxygen species (ROS), responsible for various harmful and irreversible effects for cells, tissues and DNA.

(Boc)₂-AA-OH (where AA means the cross-linking agent, arginine or ornithine) 1 g, Lipochroman (if desired 1.5 equivalents) and DIC N, N′-Diisopropylcarbodiimide (if desired 1.5 equivalents) were dissolved in 70 ml of dichloromethane (CH₂Cl₂) and the solution was stirred, for example for a time between 30 and 90 minutes, if desired 1 h at a temperature lower than 5° C., for example about 0° C. 1.5 equivalents of 4-(N, N-dimethylamino)-pyridine catalyst (DMAP) were dissolved in 20 ml of DCM and added to the solution, dropwise if desired.

The reaction mixture was left under stirring, if desired at room temperature overnight or for a time exceeding 6 hours, for example covered with aluminium foil and monitored by TLC the following day.

After a time greater than 15 h, for example 20h, the reaction was complete, so that the solvent could be removed, if desired in vacuo.

The product was extracted, for example with ethyl acetate and washed with citric acid 10%, NaHCO₃ 5% and a saturated solution of NaCl (twice).

The recovered organic phase was anhydrified with NaSO₄, filtered and concentrated. Subsequently, the Boc protecting group was removed in an acid medium (HCl in 4M dioxane) at room temperature for 1 h. The solution was concentrated by evaporation under vacuum and the product precipitated with diethyl ether. The yield for the deprotection phase is 80-95%.

To eliminate traces of mono-protected intermediates, the product was recrystallized in ethyl acetate. The new synthesized prodrugs, characterized by ¹H-NMR and mass spectroscopy, show high purity. The same are used for the crosslinking of the linear HA polymer according to the above procedure to obtain a prodrug of polymeric crosslinked (product C) and (product D).

The crosslinking products (A), (B), (C) and (D) were then evaluated with the analytical techniques deemed suitable and discussed below.

The general formula of cross-linked hyaluronic acid, with inter-chain cross-linking obtainable with a process according to the present invention, is reported below.

This acid is the subject of the present invention also as such and therefore independently of the obtaining process. The relative formula is shown below, in which X, Y, Z, n have the meaning indicated above.

According to a non-limiting example of the present invention, the formula below provides that: X═OH, n=0-1; Y═NH, n=0-1; Z═NH, n=0-1 or another example provides that: X═COOR, n=0-1; Y═NH, n=0-1; Z═NH, n=0-1, R═H, alkyl.

When n=0, the relative portion of the molecule is not present and therefore, for example, COOH is directly linked to the base backbone of the molecule.

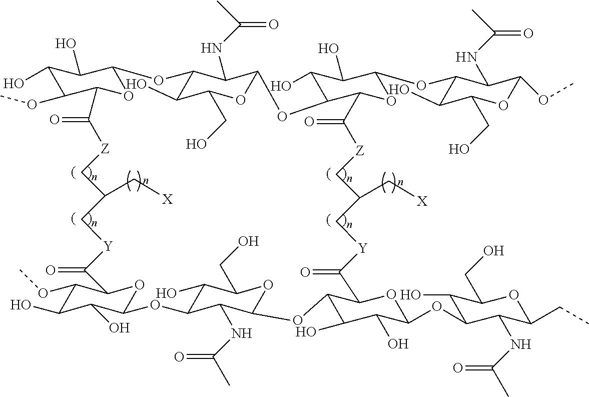

Instead, the general formula of cross-linked hyaluronic acid, with intrachain cross-linking, obtainable with a process according to the present invention is reported below.

This acid is the subject of the present invention also as such and therefore independently of the obtaining process. Also in this case, X, Y, Z, n have the meaning indicated above.

According to a non-limiting example of the present invention, the formula reported below provides that: Z═Y═NH, X═NH2, NHR, COOR, SR, R═H, alkyl, n=2-6.

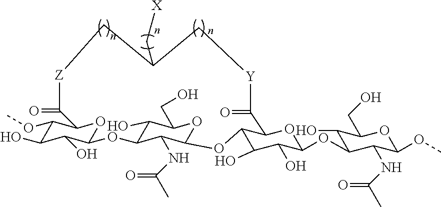

With both intra and intermolecular bonds, the result is a greater internal organization of the hyaluronic acid chains in cages of variable size given by the formation of covalent bonds and therefore the implementation of the resistance from the physical and mechanical point of view of the resulting molecule.

¹H-NMR Analysis

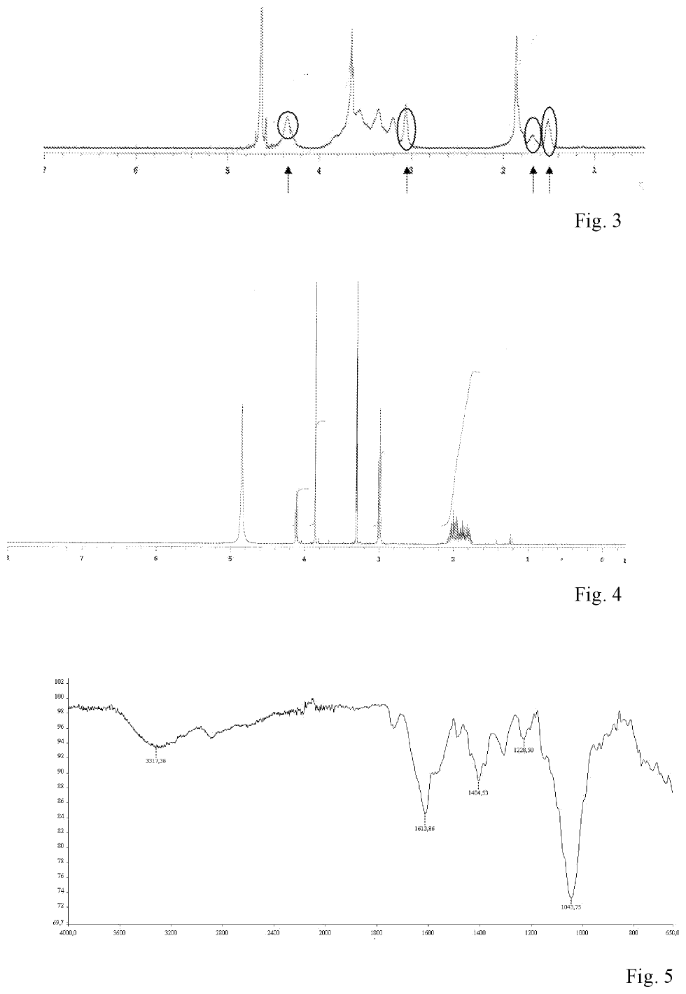

NMR spectroscopy is the most common tool for a first characterization of the chemical modification made to HA and is useful also in its quantification. The spectra of hyaluronic acid-based samples give broad signals and this is due to the viscosity of the product in solution. FIGS. 2 and 3 show the ¹H-NMR spectra of sodium hyaluronate (FIG. 2) and of HA cross-linked with arginine (HA-CL-Arg) (FIG. 3) and cross-linked with ornithine (HA-CL-Orn) (FIG. 4).

FIGS. 2 to 4 are therefore aimed at the comparative analysis of the ¹HNMR spectrum in D₂O of native HA and of the crosslinking product obtained using CDMT/NMM.

The new peaks that are detected in the spectrum of FIG. 3 are related to the methyl groups of arginine in position α (4.42 ppm), β (1.67 ppm), γ (1.52 ppm) δ (3.19 ppm) and to the methoxyl protecting group—OCH₃ (3.61 ppm). The multiplet of signals in the region from 3.3 to 4.9 ppm corresponds to the protons of the glucuronic component and N acetyl glucosamine of the hyaluronic acid.

From the calculation of the value of the integrations of the new non-overlapping peaks, in comparison with the characteristic peak at 1.9 ppm of the HA belonging to the protons of the N-acetyl CH3, it is possible to calculate the degree of derivatization which is 62%.

NMR analysis therefore provides useful information on the actual presence of the cross-linking agent in the product, but does not give indications on the nature of its presence, whether as a physical mixture or as part of a covalent bond. The characterization was then deepened by carrying out further studies capable of determining the quality of the bond.

Infrared Spectroscopy (FT-IR)

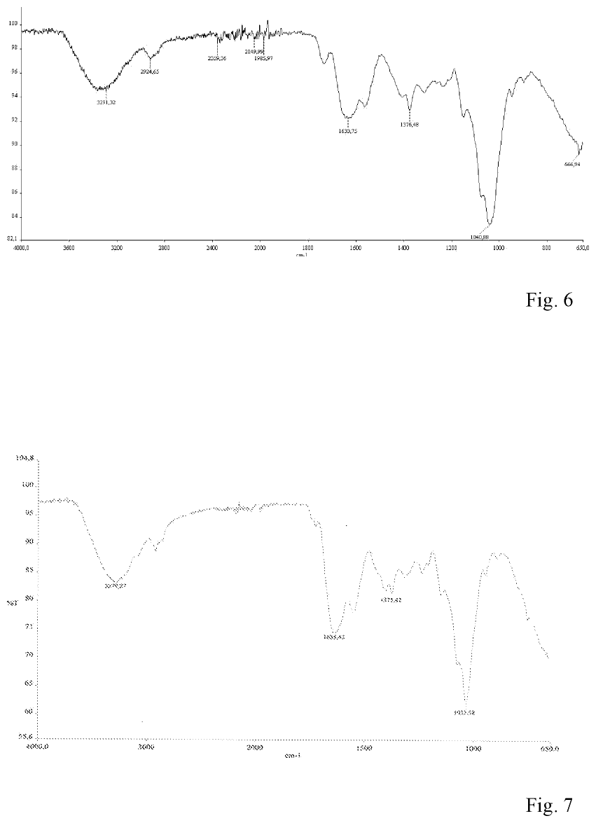

FT-IR analysis was used to characterize the chemical structure of the synthesized cross-link of HA. In this regard, in FIGS. 5 to 7 IR spectra of linear HA (FIG. 5), arginine cross-linked HA (b) (FIG. 6) and ornithine cross-linked HA (FIG. 7) are shown.
FIG. 6 shows the appearance of a band at about 1640 cm⁻¹ distinctive of an amide bond of secondary type and the simultaneous disappearance of two strong bands, in the native HA spectrum, at 1613 and 1404 cm⁻¹ of the symmetrical and asymmetric stretching of the starting carboxylate ion. The obtained formation of the amide bond is also confirmed by the increase in intensity in the NH stretching region (3350 cm⁻¹) and in the 1515 cm⁻¹ band of the NH bending. The typical signals of the distinctive groups of HA that do not take part in the reaction remain in the spectrum of the product of crosslinking.

Differential Scanning Calorimetry (DSC)

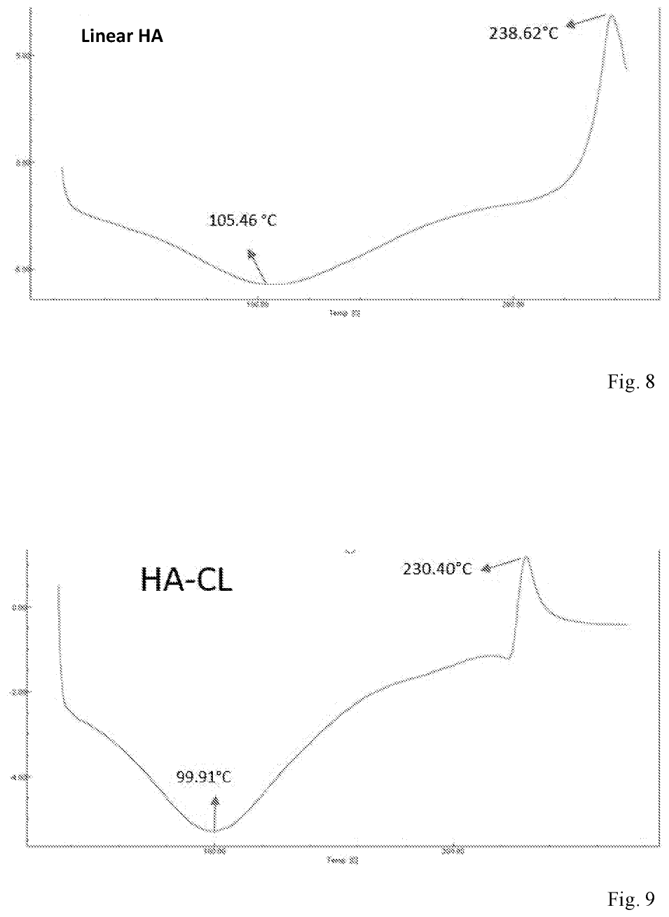

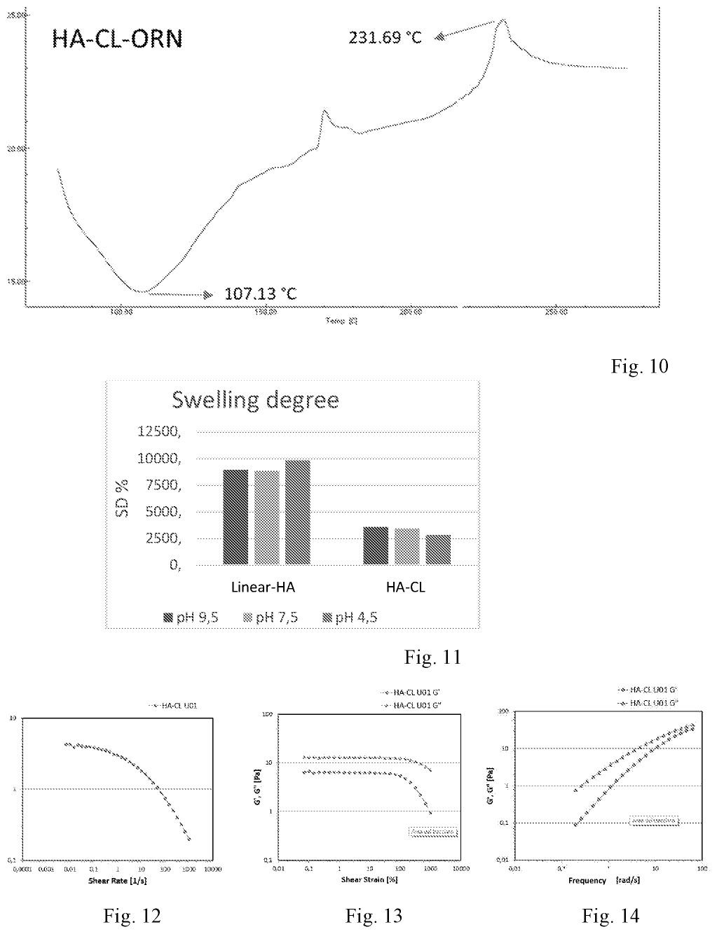

For a complete characterization, the product was subjected to calorimetric analysis by DSC. The operating principle of the DSC is based on the different temperature request between the reference and the sample as well as on the measurement of the heat absorbed or released by the system as the temperature varies when some structural modification occurs in it.
The thermograms shown in FIGS. 8 to 10 show the thermal behaviour of linear HA (FIG. 8), of HA cross-linked with arginine (FIG. 9) and of HA cross-linked with ornithine (FIG. 10).

Linear HA sample 1 shows two peaks: the first is an endothermic peak and indicates the temperature at which the sample dehydration takes place (105° C.), followed by an exothermic peak attributable to the decomposition temperature of the sample (238° C.). A similar behaviour was observed for sample 2 (HA cross-linked with arginine), but with values that tend to shift at lower temperatures: the endothermic peak is at about 100° C. followed by the exothermic peak at 230° C. The cross-linked with ornithine (sample 3) instead shows a value of the endothermic peak shifted by 2° C. above those of the linear polymer while the exothermic peak is instead in line with sample 2 and shows itself at 231° C.

By comparing the thermodynamic parameters obtained, they are attributable to an alteration of the original system. The endothermic peak is found at higher temperature values for native HA, lowering in the thermogram of the product of crosslinking 2 and rising in sample 3 even if only by a few degrees, so we could define it unchanged and this can be interpreted in calorimetric terms as an increase or decrease of the energy necessary to remove the water content in the sample. The exothermic peak has also undergone a change as the crosslinking has clearly produced a new material with a structural organization different from the original one.

| | Thermal analysis parameters | | |
|---|---|---|---|
| | Linear HA | HA-CL-Arg | HA-CL-Orn |
| Dehydration T (° C.) | 105.46 | 99.91 | 107.13 |
| Decomposition T (° C.) | 238.62 | 230.40 | 231.69 |

Physical Characterization—Swelling Measurements

The swelling test, able to quantify the water content that the sample is able to absorb, is used to study the crosslinking density of the HA hydrogel cross-linked with the crosslinking agent.
High swelling values are correlated to a lower cross-linking density: the higher the degree of cross-linking, the lower the capacity to absorb water and therefore the lower its swelling value.
FIG. 11 shows a graph indicating the degree of swelling of hyaluronic acid (linear HA) and cross-linked hyaluronic acid (HA-CL) at different pH values.

As expected, the degree of swelling obtained is significantly lower in the cross-linking product compared to linear HA and this confirms what has already been hypothesized following the DSC analysis, i.e. a decrease in the availability of carboxylic groups to form H bonds with water due to their engagement in the amide bond with the amino acid, resulting in a lower degree of swelling correlated to the lower amount of water that the sample is able to retain. The graph shown in FIG. 11 highlights the drastic drop in the swelling values of HA-CL or cross-linked hyaluronic acid in comparison with the values shown by linear hyaluronic acid and an inversion of pH-dependent behavior.

The swelling was calculated with the following formula which relates the weight of the swollen molecule to the weight of the dry sample:

SD %=WS/WD×100

wherein WS is the weight deriving on 0,100 g of swollen product after an immersion time of 24 h at room temperature at different pH and WD is the weight of the dry product.

Rheological Analysis

Rheological studies are essential to evaluate the effect of the modification made to the starting molecule in support of products with optimal characteristics that can be used in various fields of biomedical applications.

In the case in question, the degree of crosslinking and the analysis of the rheological properties resulting from it were evaluated on the product in aqueous solution at a concentration of 20 mg/ml and conducted both in constant stress mode and in oscillatory mode.

FIGS. 12 to 14 show the rheological analysis conducted on a 2% cross-linked HA sample in conditions of continuous flow (FIG. 12) and in oscillatory flow: Amplitude sweep (FIG. 13) and Frequency sweep (FIG. 14).

As can be seen from the graphs in FIGS. 12 to 14, the HA-CL product exhibits shear-thinning properties: the viscosity of the sample decreases as the speed of the applied force increases.

Conducting the study in oscillatory stress allowed the determination of the elastic modulus (G′) and the viscous modulus (G″) where the first provides information on the elasticity of the material while the second reflects the viscous behaviour.

In FIGS. 12 to 14 it is possible to see that the behaviour of the product is that typical of a viscous solution, with the viscous modulus always higher than the elastic modulus. The observed viscosity profile may indicate a possible degradation process following the chemical purification and freeze-drying steps or more concretely this behaviour could be linked both to the molecular weight of the molecule produced and to the molecular weight of the cross-linking agent used.

The elastic modulus of these materials tends, in fact, to decrease in a way that is inversely proportional to the molecular weight of the cross-linking molecule.

Scanning Electron Microscopy (SEM)

For the morphological characterization of the crosslink, the lyophilized sample was subjected to SEM microscopy analysis thanks to which it was possible to highlight the transformation of HA which, from a filamentous and irregular structure typical of linear hyaluronic acid, becomes, following the process of cross-linking, a polymer with a more ordered and highly porous structure.

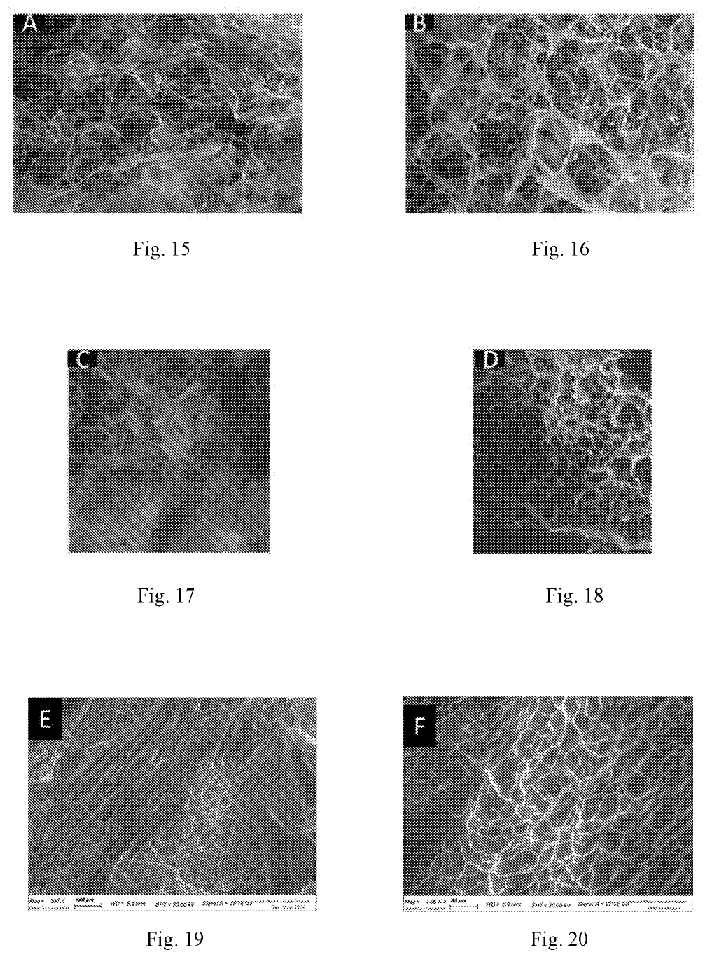

FIGS. 15 to 20 show SEM images of linear HA (A and C), of HA cross-linked with Arginine (B and D) and cross-linked with ornithine (E and F).

As it is possible to observe from the scanning electron microscopy images, the crosslinking brings about an internal organization of the polymeric matrix forming real cages of homogeneous dimensions that make the HA derivative a vehicle for drugs (or at least one molecule indicated above) able to be included and released in successive and gradual moments as a result of polymer degradation.

The subject of the present invention is also the topical and possibly systemic use by oral ingestion of cross-linked hyaluronic acid according to the present invention.

In this regard, the innovation consists in the properties possessed by cross-linked hyaluronic acid of including active ingredients, in the greater resistance to hyaluronidases and in the best elastic and film-forming properties.

It will therefore be understood that thanks to the present invention it is possible to obtain a new process for the synthesis of cross-linked hyaluronic acid, which allows to produce hyaluronic acid with a prolonged half-life when injected or inserted in the site of use, for example in the skin of a man or a woman. and which allows the insertion of biologically active substances by physical inclusion in the cages or by covalent bonding with the linker.

Moreover, the cross-linked hyaluronic acid thus obtained is highly biocompatible with the body tissues of implantation or injection.

1. Cross-linked hyaluronic acid synthesis process which comprises the following steps:
preparing a predetermined quantity of hyaluronic acid and at least one cross-linking agent of an amino acid nature, said hyaluronic acid being in the form of a linear polymer or already partially cross-linked, and
cross-linking said hyaluronic acid by said at least one cross-linking agent by means of amide bonds made on the carboxylic portions of linear chains of said linear polymer or of the already partially cross-linked polymer.

2. Process according to claim 1, wherein said at least one cross-linking agent comprises at least a first biologically active molecule compatible with the tissues of the human organism and with an arrangement provided with at least 2 functionalizable portions, the cross-linking being carried out by means of the homobifunctional portion of the molecule given by 2 free or salified amino groups, one of which is the end of the side chain (ω) and the other in alpha (α).

3. Process according to claim 2, wherein said at least one first biologically active molecule comprises at least one amino acid or a synthesis substrate thereof.

4. Process according to claim 3, wherein said at least one first biologically active molecule comprises at least one substrate of the proline synthesis.

5. Process according to claim 4, wherein said at least one first biologically active molecule is selected from the group consisting of arginine, ornithine or mixtures or derivatives thereof, for example arginine and/or ornithine methyl ester.

6. Process according to any one of claim 2, wherein said at least one first biologically active molecule comprises at least 3 functionalizable portions, the third function being a carboxyl group, and wherein said process comprises, before or after cross-linking, a step of derivatization of said at least one first biologically active molecule which consists in the connection of at least one second biologically active molecule at said carboxylic group, so that said at least one first biologically active molecule has the function both of cross-linking to form cross-links between different linear hyaluronic acid chains or between two different points of the same chain, and as a linker for constituting a prodrug due to the effect of a covalent or saline bond to said at least one second biologically active molecule.

7. Process according to claim 6, wherein said first biologically active molecule has a formula as indicated below: 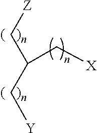
X═—OH, —NH2, —NRH, —SH, —COOH
Y═—OH, —NH2, —NRH, —SH, —COOH
Z═—OH, —NH2, —NRH, —SH, —COOH
R=alkyl, aryl, —NH2, —NHR, —CO-Q
Q=R, —OH, —SH
wherein n is any integer number of possible groups and/or atoms such as for example —CH2-, —CHR, —CRR—, —O—, —NRR—, —S—, etc.

8. Process according to claim 6, wherein said at least one second biologically active molecule comprises a drug or a pharmacologically active molecule or a healthy molecule, such as for example a molecule having positive effects on the maintenance of a good state of health, for example a vitamin, or a cosmetic ingredient or a food ingredient.

9. Process according to claim 6, wherein said at least one second biologically active molecule contains in its structure a functional group capable of reacting with said carboxylic group of said at least one first biologically active molecule, such as for example NH₂, OH, COOH, NHR, SH, CO—NH2, CO—NHR, NH—NH2, in cui R=alchile, arile, —NH2, —CO-Q e Q=R, —OH, —SH.

10. Process according to claim 1, comprising at least one step of inclusion or dispersion of a second biologically active molecule comprising a drug such as for example a pharmacologically active molecule or a healthy molecule, for example a molecule having positive effects on the maintenance of a good state of health, for example a vitamin, or a cosmetic ingredient or a food ingredient in the polymeric matrix of said hyaluronic acid before or after its cross-linking, so that said at least one second biologically active molecule comprising a drug such as for example a pharmacologically active molecule or a healthy molecule, for example a molecule having positive effects on the maintenance of a good state of health, for example a vitamin, or a cosmetic ingredient or a food ingredient, is housed in the cavities of defined size created with the cross-linking process in order to obtain a prolonged release due to the slower degradation effect brought about by the cross-linking itself.

11. Process according to claim 1, comprising before said cross-linking an initial step of activation of hyaluronic acid by derivatization with EDC, namely 1-ethyl-3-[3-(dimethylamino)-propyl]-carbodimide.

12. Process according to claim 11, wherein said EDC activates the carboxylic group of hyaluronic acid so as to form a highly reactive intermediate and wherein the process comprises an introduction step of N-hydroxy-succinimide (NHS) or 1-hydroxybenzotriazole (HOBt) arranged to prevent the reorganization of said intermediate in a stable by-product.

13. Process according to claim 11, wherein said activation step is carried out in water with pH at a value between about 4 and 5, for example at 4.75.

14. Process according to claim 1, comprising before said cross-linking an initial step of activation of hyaluronic acid by derivatization with CDMT, i.e. 2-chloro-dimethoxy-1,3,5-triazine, or as an alternative DMTMM, i.e. 4-(6-dimethoxy-1,3,5-triazin-2-yl) 4-methyl morpholin chloride, the hyaluronic acid intermediate activated with CMDT or DMTMM undergoing the nucleophilic attack of the amine group of said at least one first biologically active molecule with consequent formation of the amide bond.

15. Process according to claim 14, wherein said reaction is carried out in a mixture of water and acetonitrile solvents, for example in a 3:2 ratio, for an optimal solubilization of the reactants.

16. Process according to claim 14, wherein said activation step is carried out in the presence or with the introduction of N-methylmorpholine (NMM) to neutralize the released chloride ions.

17. Process according to claim 5, wherein the ratio between hyaluronic acid, CDMT or DMTMM and arginine or ornithine is 1:2:4, 1:3:4 or 1:3:1.5.

18. Process according to claim 1, comprising the following steps in sequence:
solubilizing sodium hyaluronate in water and leaving it to stir until a clear and agglomerate-free solution is obtained,
adding acetonitrile,
cooling the solution,
once the solution is cold, introducing CDMT or DMTMM and leaving the solution thus obtained under stirring,
after a predetermined time, introducing Arg-OMe or Orn-OMe and then NMM to neutralize the chloride ions,
leaving the obtained reaction mixture under stirring until completely cross-linked,
purifying the mixture from amines and excess reagents.

19. Process according to claim 6, wherein the derivatization of said at least one first biologically active molecule comprises an initial protection step of said at least a first molecule by means of Boc groups on the amino functionalities of said at least one first molecule.

20. Process according to claim 19, wherein said at least one first molecule is esterified with Lipocrhoman®, a compound having the following formula 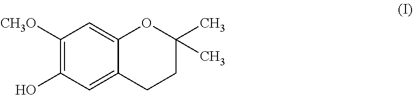
this compound being capable of capturing reactive nitrogen species (RNS) and reactive oxygen species (ROS), responsible for various harmful and irreversible effects on cells, tissues and DNA.

21. Process according to claim 19, comprising the following steps:
(Boc)₂-AA-OH and Lipochroman and DIC N,N′-Diisopropylcarbodiimide were dissolved in dichloromethane and the solution thus obtained was stirred,
dissolving the catalyst 4-(N,N-dimethylamino)-pyridine (DMAP) in DCM and adding what is thus obtained to the previous solution, obtaining a reaction mixture,
leaving the reaction mixture under stirring and then removing the solvent,
extracting the product obtained with ethyl acetate and washing it if desired with citric acid and a saturated solution of NaCl,
recovering the organic phase,
removing the Boc protector group in an acid environment,
concentrating in vacuum the product obtained thus far and precipitating it with diethyl ether, if necessary, to eliminate traces of mono protected intermediates, recrystallizing the product obtained previously in ethyl acetate.

22. Cross-linked hyaluronic acid obtained by a process according to claim 1.

23. Cross-linked hyaluronic acid having the following formula: 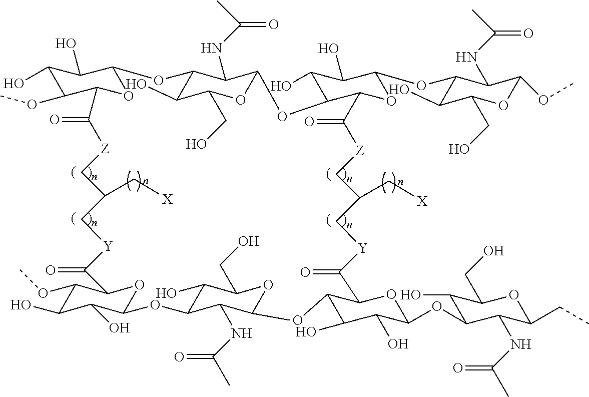
wherein
X═—OH, —NH2, —NRH, —SH, —COOH
Y═—OH, —NH2, —NRH, —SH, —COOH
Z═—OH, —NH2, —NRH, —SH, —COOH
R=alkyl, aryl, —NH2, —NHR, —CO-Q
Q=R, —OH, —SH
wherein n is any integer number of possible groups and/or atoms such as for example —CH2-, —CHR, —CRR—, —O—, —NRR—, —S—, etc., or wherein X═OH, n=0-1; Y═NH, n=0-1; Z═NH, n=0-1 or wherein X═COOR, n=0-1; Y═NH, n=0-1; Z═NH, n=0-1, R═H, alkyl.

24. Cross-linked hyaluronic acid having the following formula: 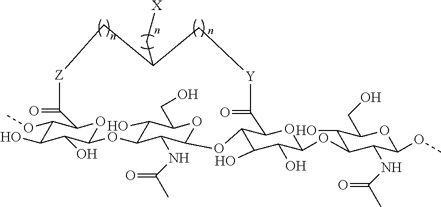
wherein
X═—OH, —NH2, —NRH, —SH, —COOH
Y═—OH, —NH2, —NRH, —SH, —COOH
Z═—OH, —NH2, —NRH, —SH, —COOH
R=alkyl, aryl, —NH2, —NHR, —CO-Q
Q=R, —OH, —SH
wherein n is any integer number of possible groups and/or atoms such as for example-CH2-, —CHR, —CRR—, —O—, —NRR—, —S—, etc., or wherein Z═Y═NH, X═NH2, NHR, COOR, SR, R═H, alkyl, n=2-6.

25. Cross-linking agent comprising a biologically active molecule with a tridentate arrangement, i.e. provided with 3 functionalizable portions, said cross-linking agent having a formula as indicated below: 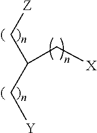
X═—OH, —NH2, —NRH, —SH, —COOH
Y═—OH, —NH2, —NRH, —SH, —COOH
Z═—OH, —NH2, —NRH, —SH, —COOH
R=alkyl, aryl, —NH2, —NHR, —CO-Q
Q=R, —OH, —SH
wherein n is any integer number of possible groups and/or atoms such as for example-CH2-, —CHR, —CRR—, —O—, —NRR—, —S—, etc.

26. Topical or systemic use for oral ingestion of cross-linked hyaluronic acid according to claim 22.
